(19) United States
(12) Patent Application Publication
(10) Pub. No.: US 2026/0294965 A1
(43) Pub. Date: Oct. 1, 2026

(54) Method for Treating Liver Cancer Using Cell-Immune Agent Composition Delivered via Hepatic Artery

(71) Applicant: NATIONAL DEFENSE MEDICAL UNIVERSITY, Taipei City (TW)

(72) Inventors: Bing-Heng YANG, Taipei City (TW); Wei-Chen HUANG, Taipei City (TW)

(21) Appl. No.: 19/276,526

(22) Filed: Jul. 22, 2025

(30) Foreign Application Priority Data
Mar. 28, 2025 (TW) 114112176

(51) Int. Cl. A61K 35/15; A61K 35/17; A61K 39/00; A61K 39/395; A61P 35/00

(52) U.S. Cl. CPC A61K 35/15; A61K 35/17; A61K 39/39558; A61P 35/00; A61K 2039/505; A61K 2039/54

(57) ABSTRACT

The present invention disclosed a method for treating liver cancer in a subject in need thereof. The method includes administering autologous immune cells and immune checkpoint inhibitors to a subject via hepatic artery injection.

### BACKGROUND OF THE INVENTION

### 1. Field of the Invention

The present invention provides a method for treating liver cancer, specifically involving hepatic artery infusion of autologous immune cells in combination with immune checkpoint inhibitors.

### 2. Description of the Prior Art

Hepatocellular carcinoma (HCC), which accounts for about 90% of primary liver cancers, is one of the most lethal malignant tumors in the world. Unfortunately, around 10~40% of HCC patients at the time of diagnosis, defined as Barcelona Clinic Liver Cancer (BCLC) advanced stage, have very limited treatment options. Unsuitable for curable surgical resection, liver transplantation, or radiofrequency ablation, those patients have a particularly poor prognosis and the median survival is only prolonged a few months by systemic treatment with tyrosine kinase inhibitors.

Recently, the Taiwan Liver Cancer Association and the Gastroenterological Society of Taiwan have established a management consensus guideline for systemic treatment of HCC. Immunotherapy using anti-programmed death ligand 1 (PD-L1) or anti-programmed cell death 1 (PD-1) to rescue and revitalize exhausted T cells has been adopted as a new strategy for patients with advanced HCC in Taiwan. However, there are some limitations of immune checkpoint blockade (ICB). For example, the treatment response rate of anti-PD-1 for HCC is generally lower than 20%. Therefore, combining multiple therapeutic modalities to enhance treatment responses is urgently needed for patients with advanced HCC.

### SUMMARY OF THE INVENTION

In order to improve the limitations of conventional therapies for HCC, the present invention provides a method for treating liver cancer in a subject in need thereof. The method comprises the following steps: administering a therapeutically effective amount of autologous immune cells to the subject, and administering a therapeutically effective amount of an immune checkpoint inhibitor (ICI) to the subject; wherein the therapeutically effective amount of the autologous immune cells and the therapeutically effective amount of the immune checkpoint inhibitor are administered to the subject via hepatic artery infusion.

In certain embodiments, the therapeutically effective amount of the autologous immune cells and the therapeutically effective amount of the immune checkpoint inhibitor are administered to the subject simultaneously.

In certain embodiments, a portion of the therapeutically effective amount of the autologous immune cells is administered to the subject first, the therapeutically effective amount of the immune checkpoint inhibitor is administered to the subject secondly, and then a remaining portion of the therapeutically effective amount of the autologous immune cells is administered to the subject. In certain embodiments, the portion of the therapeutically effective amount of the autologous immune cells is one quarter to three quarters of the therapeutically effective amount. In certain embodiments, the remaining portion of the therapeutically effective amount of the autologous immune cells is three quarters to one quarter of the therapeutically effective amount.

In certain embodiments, the therapeutically effective amount of the autologous immune cells is isolated through leukapheresis. In certain embodiments, the leukapheresis is configured to isolate mononuclear cells having a density of between 1.055 to 1.08 g/mL.

In certain embodiments, the therapeutically effective amount of the autologous immune cells is administered within 12 hours after the autologous immune cells are isolated.

In certain embodiments, the therapeutically effective amount of the autologous immune cells comprises dendritic cells and lymphocytes.

In certain embodiments, the therapeutically effective amount of the autologous immune cells comprises at least 1×10⁶ dendritic cells.

In certain embodiments, the therapeutically effective amount of the autologous immune cells comprises at least 1×10⁸ lymphocytes.

In certain embodiments, the therapeutically effective amount of the autologous immune cells comprises at least 1×10⁶ dendritic cells and at least 1×10⁸ lymphocytes.

In certain embodiments, the immune checkpoint inhibitor is a programmed death-ligand 1 (PD-L1) inhibitor, wherein the PD-L1 inhibitor is used to reduce immunosuppressive factors in the tumor microenvironment and reduce the expression of PD-L1 on antigen presenting cells (APCs), thereby activating the anti-cancer ability of T cells.

In certain embodiments, the therapeutically effective amount of the immune checkpoint inhibitor comprises around 1 to 50 mg/mL of PD-L1 inhibitor.

In certain embodiments, the therapeutically effective amount of the immune checkpoint inhibitor comprises 1~5,000 mg of PD-L1 inhibitor.

In certain embodiments, the subject has liver cancer. In certain embodiments, the subject received radiotherapy prior to the administration of autologous immune cells and the immune checkpoint inhibitor.

Unlike cell therapy by infusion of immune cells collected from other people, the present invention provides a regimen of administration with self-dendritic cells/lymphocytes, and this method is highly tolerable without allogeneic adverse responses. In addition, the method of the present invention combines immune-oncology therapy (IO) and autologous DC/lymphocyte administration to successfully boost anti-tumor activity resulting in remarkable refractory tumor shrinkage.

These and other aspects will become apparent from the following description of the preferred embodiment taken in conjunction with the following drawings.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIG. 1 shows the transverse plane computed tomography (CT) images of Patient 1 in one embodiment of the present invention, taken before treatment (left panel) and after treatment (right panel). Arrows highlight treatment-induced tumor shrinkage and destruction of the tumor parenchyma.

FIG. 2 shows the transverse plane computed tomography (CT) images of Patient 1 in one embodiment of the present invention, taken before treatment (left panel) and after treatment (right panel). Arrows highlight treatment-induced disruption of tumor-associated thrombosis and improved patency of the hepatic portal vein.

FIG. 3 shows the coronal plane computed tomography (CT) images of Patient 2 in one embodiment of the present invention, taken before treatment (left panel) and after treatment (right panel). Arrows highlight treatment-induced tumor shrinkage and extensive necrosis of the tumor parenchyma.

FIG. 4 shows the coronal plane computed tomography (CT) images of Patient 2 in one embodiment of the present invention, taken before treatment (left panel) and after treatment (right panel). Arrows highlight treatment-induced tumor shrinkage and extensive necrosis of the tumor parenchyma.

FIG. 5 shows transverse computed tomography (CT) images of Patient 2 in one embodiment of the present invention, taken before treatment (left panel) and six months after treatment (right panel). The region enclosed by black arrows with a white border indicates the infiltrative liver tumor prior to treatment, while the region marked by gray arrows with a white border shows the same area after 6 months of treatment, demonstrating tumor shrinkage and parenchymal necrosis.

FIG. 6 shows longitudinal changes in PIVKA-II levels in Patient 2, in one embodiment of the present invention, recorded at baseline (pre-treatment), after 3 months of treatment, and after 6 months of treatment.

FIG. 7 shows flow cytometry analysis of activated dendritic cells from patient 2 in one embodiment of the present invention, conducted at baseline (pre-treatment), after 6 months of treatment.

FIG. 8 shows flow cytometry analysis of activated T cells from patient 2 in one embodiment of the present invention, conducted at baseline (pre-treatment), after 6 months of treatment.

### DETAILED DESCRIPTION OF THE PREFERRED EMBODIMENT

It should be understood that both the foregoing general description and the following detailed description are exemplary and explanatory but are not restrictive of the invention as claimed. Certain details of one or more embodiments of the invention are set forth in the description below. Other features or advantages of the present invention will be apparent from the non-exhaustive list of representative examples that follows, and also from the appending claims.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well-known in the art. Generally, nomenclatures used in connection with, and techniques of biochemistry, enzymology, molecular and cellular biology, microbiology, clinical medicine, immunology, genetics, and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated.

It should also be understood that, unless clearly indicated to the contrary, in any methods claimed herein that include more than one step or act, the order of the steps or acts of the method is not necessarily limited to the order in which the steps or acts of the method are recited.

As used herein, the singular form “a”, “an”, and “the” includes plural references unless indicated otherwise. For example, “an” excipient includes one or more excipients.

As used herein, the term “or” is used interchangeably with the term “and/or” unless the context clearly indicates otherwise.

As used interchangeably herein, “around”, “about” and “approximately” shall generally mean plus or minus 10% of the numerical value of the number with which it is being used. Therefore, about 1% means in the range of 0.9% to 1.1%. Numerical quantities given herein are approximate, meaning that the term “around”, “about” or “approximately” can be inferred if not expressly stated.

As used herein, the phrase “comprising” is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

In one aspect, the present invention provides a method for treating liver cancer in a subject in need thereof. The method comprises the following steps: administering a therapeutically effective amount of autologous immune cells to the subject, and administering a therapeutically effective amount of an ICI to the subject; wherein the therapeutically effective amount of the autologous immune cells and the therapeutically effective amount of the immune checkpoint inhibitor are administered to the subject via hepatic artery infusion.

The principle of hepatic arterial injection involves directly injecting a high concentration of drugs (or formulations) into the hepatic artery to enhance therapeutic efficacy and reduce systemic side effects. Normal liver tissue primarily relies on the portal vein for its blood supply and nutrient intake. In contrast, liver cancer tumors mainly depend on the hepatic artery for their nutrient supply. Therefore, delivering anticancer drugs through the hepatic artery can effectively concentrate the drug's action on liver cancer cells, with minimal impact on the surrounding normal liver tissue. In certain embodiments, the hepatic arterial injection comprises hepatic arterial infusion, which involve using a drip pump to control the infusion rate (80 to 90 ml/hour).

In certain embodiments, the therapeutically effective amount of the autologous immune cells and the therapeutically effective amount of the immune checkpoint inhibitor are administered to the subject simultaneously.

In certain embodiments, a portion of the therapeutically effective amount of the autologous immune cells is administered to the subject first, the therapeutically effective amount of the immune checkpoint inhibitor is administered to the subject secondly, and then a remaining portion of the therapeutically effective amount of the autologous immune cells is administered to the subject. In certain embodiments, the portion of the therapeutically effective amount of the autologous immune cells is one quarter to three quarters of the therapeutically effective amount. In certain embodiments, the remaining portion of the therapeutically effective amount of the autologous immune cells is three quarters to one quarter of the therapeutically effective amount.

In certain embodiments, the autologous immune cells are isolated via leukapheresis. In certain embodiments, the leukapheresis is configured to isolate mononuclear cells having a density of between 1.055 to 1.08 g/mL.

In certain embodiments, the therapeutically effective amount of the autologous immune cells is administered within 12 hours after the autologous immune cells are isolated. For example, the autologous immune cells are administered within 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, or 12 hours, after the autologous immune cells are isolated.

In certain embodiments, the therapeutically effective amount of the autologous immune cells comprises dendritic cells and lymphocytes.

In certain embodiments, the therapeutically effective amount of the autologous immune cells comprises at least 1×10⁶ dendritic cells. For example, the therapeutically effective amount of the autologous immune cells comprises at least 1×10⁶, at least 2.5×10⁶, at least 5×10⁶, at least 7.5×10⁶, at least 1×10⁷, at least 2.5×10⁷, at least 5×10⁷, at least 7.5×10⁷, at least 1×10⁸, at least 2.5×10⁸, at least 5×10⁸, at least 7.5×10⁸, at least 1×10⁹, at least 2.5×10⁹, at least 5×10⁹, at least 7.5×10⁹, or at least 1×10¹⁰ dendritic cells.

In certain embodiments, the therapeutically effective amount of the autologous immune cells comprises at least 1×10⁸ lymphocytes. For example, the therapeutically effective amount of the autologous immune cells comprises at least 1×10⁸, at least 2.5×10⁸, at least 5×10⁸, at least 7.5×10⁸, at least 1×10⁹, at least 2.5×10⁹, at least 5×10⁹, at least 7.5×10⁹, or at least 1×10¹⁰, at least 2.5×10¹⁰, at least 5×10¹⁰, at least 7.5×10¹⁰, at least 1×10¹⁰, at least 2.5×10¹¹, at least 5×10¹¹, at least 7.5×10¹¹, at least 1×10¹² lymphocytes.

In certain embodiments, the therapeutically effective amount of the autologous immune cells comprises at least 1×10⁶ dendritic cells and at least 1×10⁸ lymphocytes. For example, the therapeutically effective amount of the autologous immune cells comprises at least 1×10⁶, at least 1×10⁷, at least 1×10⁸, at least 1×10⁹, or at least 1×10¹⁰ dendritic cells and at least 1×10⁸, at least 1×10⁹, at least 1×10¹⁰, at least 1×10¹¹, or at least 1×10¹² lymphocytes.

In certain specific embodiments, the immune checkpoint inhibitor is a PD-L1 inhibitor.

In certain embodiments, the therapeutically effective amount of the immune checkpoint inhibitor comprises around 1 to 50 mg/mL of PD-L1 inhibitor. For example, the therapeutically effective amount of the immune checkpoint inhibitor comprises around 1 mg/mL, 2.5 mg/mL, 5 mg/mL, 7.5 mg/mL, 10 mg/mL, 12.5 mg/mL, 15 mg/mL, 17.5 mg/mL, 20 mg/mL, 22.5 mg/mL, 25 mg/mL, 27.5 mg/mL, 30 mg/mL, 32.5 mg/mL, 35 mg/mL, 37.5 mg/mL, 40 mg/mL, 42.5 mg/mL, 45 mg/mL, 47.5 mg/mL, or 50 mg/mL of PD-L1 inhibitor.

In certain embodiments, the therapeutically effective amount of the immune checkpoint inhibitor comprises 1~5,000 mg of PD-L1 inhibitor. For example, the therapeutically effective amount of the immune checkpoint inhibitor comprises around 1 mg, 10 mg, 50 mg, 100 mg, 250 mg, 500 mg, 1,000 mg, 1,500 mg, 2,000 mg, 2,500 mg, 3,000 mg, 3,500 mg, 4,000 mg, 4,500 mg, or 5,000 mg of PD-L1 inhibitor.

In certain embodiments, the subject has liver cancer. In certain embodiments, the subject received radiotherapy prior to the administration of autologous immune cells and the immune checkpoint inhibitor.

As used herein, the term “treat,” “treating,” or “treatment” encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, “an effective amount,” “a sufficient amount,” and “a therapeutically effective amount,” which can be used interchangeably, of a substance is that amount sufficient to effect beneficial or desired results, including clinical results. Specifically, it refers to a dosage sufficient to alleviate symptoms of HCC, to induce tumor destruction and shrinkage in HCC, to decrease the indication of a tumor marker for liver cancer (such as PIVKA-II), to increase the proportions of activated CD4⁺ T cells and activated CD8⁺ T cells in a patient, and/or to decrease the number of circulating tumor cells (CTCs),

As used herein, the term “portion” refers to a proportion of the total dosage of a reagent, in the case of alternating infusion of the reagent and another reagent. The portion of the total dosage of the reagent is adjusted based on clinical requirements. The portion may be one-quarter (¼), one-third (⅓), one-half (½), two-thirds (⅔), three-quarters (¾), or any other appropriate proportion of the total dosage. As used herein, the term “remaining portion” generally refers to the residual part of the dosage remaining after a portion has been used or removed relative to the original total volume.

As used herein, the term “autologous immune cells” generally refers to cells with immune function isolated from an individual's own body. The immune cells include, but are not limited to, dendritic cells, macrophages, T cells, B cells, and natural killer cells. As used herein, the term “mononuclear cells with a density of 1.055 to 1.08 g/mL” includes, but is not limited to, mononuclear cells, dendritic cells, macrophages, T cells, B cells, natural killer cells, and mononuclear cell-derived macrophage precursors.

As used herein, the term “leukapheresis” refers to a type of blood component separation technique in which an apheresis machine selectively separates mononuclear cells (e.g., T cells, mononuclear cells, etc.) from an individual's blood. These cells are often subsequently reinfused or used for further processing. This technique primarily relies on blood centrifugation and flow separation methods to precisely collect the target cells.

As used herein, the term “immune checkpoint” refers to a key molecule in the human body that regulates the immune system, primarily functioning to prevent excessive T cell activation and thereby avoid autoimmune responses. As used herein, the term “immune checkpoint inhibitor” refers generally to drugs that inhibit immune checkpoints, including but not limited to PD-L1 inhibitors, PD-1 (Programmed Cell Death Protein 1) inhibitors, and CTLA-4 (Cytotoxic T-Lymphocyte Antigen 4) inhibitors, etc. The mechanism of action lies in blocking immunosuppressive signaling through immune checkpoint pathways within the tumor microenvironment, thereby restoring the subject's immune surveillance and enabling the recognition and elimination of tumor cells, ultimately resulting in an anti-tumor effect.

As used herein, the term “PD-1 inhibitor” refers generally to drugs that inhibit PD-1, including but not limited to Pembrolizumab (sold under the brand name Keytruda®) and Nivolumab (sold under the brand name Opdivo®). As used herein, the term “PD-L1 inhibitor” refers generally to drugs that inhibit PD-L1, including but not limited to Atezolizumab (sold under the brand name Tecentriq®) and Durvalumab (sold under the brand name Imfinzi®).

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### Example 1. Clinical Study Case 1

In this Example, a liver cancer patient (Patient 1) had received radiotherapy prior to the treatment described herein. Autologous immune cells isolated from Patient 1, along with an immune checkpoint inhibitor, were administered to the patient. The therapeutic efficacy was then evaluated by assessing the improvement of the tumor condition through computed tomography (CT) imaging. The study was reviewed and approved by Tri-Service General Hospital (Taipei, Taiwan).

Mononuclear cells with a density of 1.055 to 1.08 g/mL were isolated from the peripheral blood of Patient 1 via leukapheresis to obtain high concentrations of dendritic cells and lymphocytes. Compared to the peripheral blood of Patient 1, the autologous immune cells obtained through leukapheresis had a white blood cell content increased by about 10.11 times, a lymphocyte content increased by about 34.4 times, and a mononuclear cell content increased by about 43.1 times. As the number of white blood cells and red blood cells of the patient changes, the volume of the autologous immune cells isolated each time may vary. In general, the volume of the autologous immune cells collected each time was about 150 to 200 ml.

Durvalumab (sold under the brand name Imfinzi®) was used as the immune checkpoint inhibitor. A single dose of around 120 mg of Durvalumab (with the concentration of around 5 mg/mL) was administered to Patient 1 each time.

The autologous immune cells isolated from Patient 1 through leukapheresis were administered to Patient 1 within 2 hours after the isolation. For each treatment session, approximately half of the isolated autologous immune cells (about 75 to 100 ml) were first delivered to Patient 1 via hepatic artery infusion. This was followed by the administration of approximately 120 mg of Durvalumab (about 20 to 25 ml), and then the remaining portion of the immune cells (the other half, about 75 to 100 ml) was administered. Patient 1 underwent three (3) treatment sessions, with an interval of 3 to 4 weeks between each session. The patient's cancer status was subsequently monitored to assess therapeutic response.

### Computed Tomography (CT) Imaging

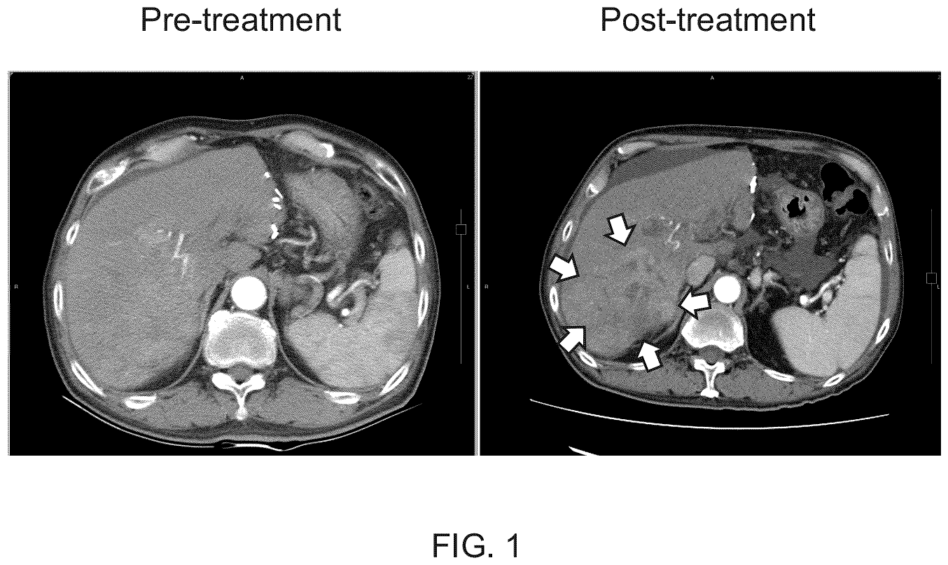

Computed tomography (CT) was used to evaluate the efficacy of the treatment. Tumor liquefaction refers to necrosis within the tumor, where tumor cells die due to insufficient blood supply or other factors, resulting in the formation of liquefied regions. In clinical practice, tumor liquefaction can serve as an indicator of tumor destruction. As shown in FIG. 1, compared to the pre-treatment liver tissue (left panel), the liver tissue of Patient 1 at three weeks after the first treatment session exhibited distinct mottled gray-black areas—non-enhancing hypodense regions within the portal vein (indicated by arrows in the right panel)—indicating tumor liquefaction in the hepatic parenchyma. These results demonstrate that the method described in the present invention effectively induces necrosis in hepatocellular carcinoma, thereby showing therapeutic efficacy against liver cancer.

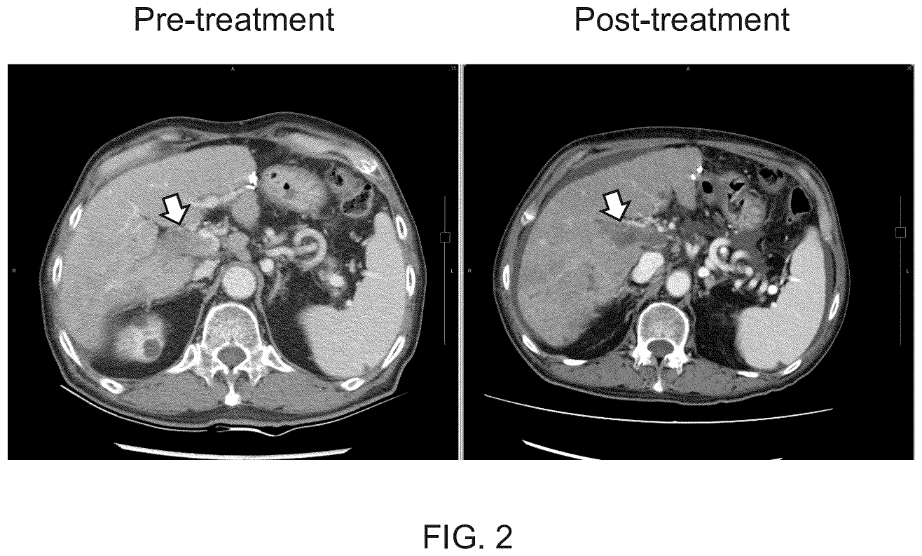

As shown in FIG. 2, prior to the treatment, Patient 1 exhibited evident tumor thrombosis in the hepatic vein (indicated by the arrow in the left panel). Following three treatment sessions, marked tumor liquidation was observed in the hepatic vein (indicated by the arrow in the right panel), indicating that the method described in the present invention effectively induces necrosis of hepatocellular carcinoma and demonstrates therapeutic efficacy against liver cancer.

### Example 2: Clinical Study Case 2

In this Example, a liver cancer patient (Patient 2) had received radiotherapy prior to the treatment described herein. Autologous immune cells isolated from Patient 2, along with an immune checkpoint inhibitor, were administered to the patient. The therapeutic efficacy was then evaluated by assessing the improvement of the tumor condition through CT imaging. In addition, CTCs and Protein induced by Vitamin K absence or antagonists-II (PIVKA-II) levels in peripheral blood were measured as biomarkers to verify the therapeutic efficacy. Immune cell activation profiles were analyzed by flow cytometry. The study was also reviewed and approved by Tri-Service General Hospital (Taipei, Taiwan).

Mononuclear cells with a density of 1.055 to 1.08 g/mL were isolated from the peripheral blood of Patient 2 via leukapheresis to obtain high concentrations of dendritic cells and lymphocytes. Compared to the peripheral blood of Patient 2, the autologous immune cells obtained through leukapheresis had a white blood cell content increased by about 8.09 times, a lymphocyte content increased by about 25.3 times, and a mononuclear cell content increased by about 32.5 times. The numbers of dendritic cells, CD4+ T cells, and CD8+ T cells in the isolated autologous immune cell are shown in Table 1 below.

TABLE 1
Numbers of autologous immune cells isolated
from Patient 2 via leukapheresis

| | Total volume | | | |
|---|---|---|---|---|
| | of autologous | Number of | Number of | Number of |
| | immune cells | dendritic | CD4+ T | CD8+ T |
| | (mL) | cells | cells | cells |
| First isolation | 144 | 1.54 × 10⁷ | 2.93 × 10⁸ | 1.13 × 10⁸ |
| Second isolation | 154 | 1.04 × 10⁷ | 2.58 × 10⁸ | 0.65 × 10⁸ |
| Third isolation | 163.4 | 1.93 × 10⁷ | 6.83 × 10⁸ | 2.38 × 10⁸ |
| Fourth isolation | 161.4 | 0.56 × 10⁷ | 6.75 × 10⁸ | 1.69 × 10⁸ |
| Fifth isolation | 173.6 | 2.27 × 10⁷ | 6.74 × 10⁸ | 4.12 × 10⁸ |

As the patient's circulating blood cell count fluctuates, the yield of autologous immune cells isolated at each collection may vary accordingly. In general, the volume of the autologous immune cells collected each time was about 150 to 200 ml.

Durvalumab (sold under the brand name Imfinzi®) was used as the immune checkpoint inhibitor. A single dose of around 120 mg of Durvalumab (with the concentration of around 5 mg/mL) was administered to Patient 2 each time.

The autologous immune cells isolated from Patient 2 through leukapheresis were administered to Patient 2 within 2 hours after the isolation. For each treatment session, approximately half of the isolated autologous immune cells (about 75 to 100 ml) were first delivered to Patient 2 via hepatic artery infusion. This was followed by the administration of approximately 120 mg of Durvalumab (about 20 to 25 ml), and then the remaining portion of the immune cells (the other half, about 75 to 100 ml) was administered. Patient 2 underwent five (5) treatment sessions, with an interval of 3 to 4 weeks between each session. The patient's cancer status was subsequently monitored to assess therapeutic response.

### 2.1 Computed Tomography (CT) Imaging

CT was used to evaluate the efficacy of the treatment. Tumor liquefaction refers to intratumoral necrosis, wherein tumor cells die due to inadequate blood supply or other factors, leading to the formation of liquefied regions. Clinically, tumor liquefaction is considered an indicator of effective tumor destruction.

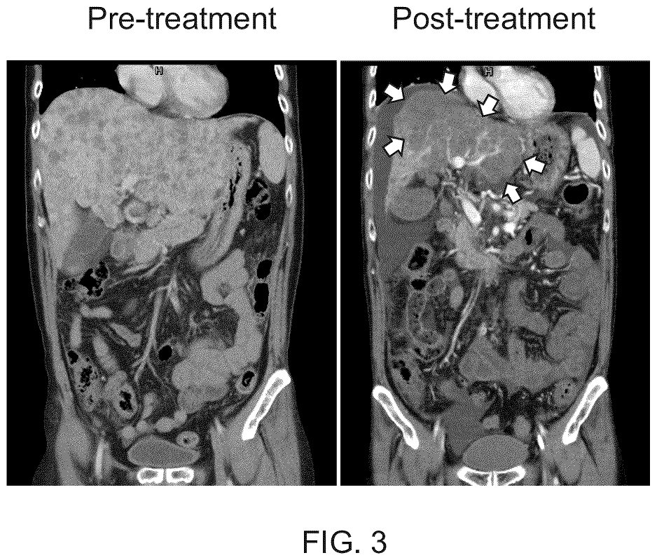

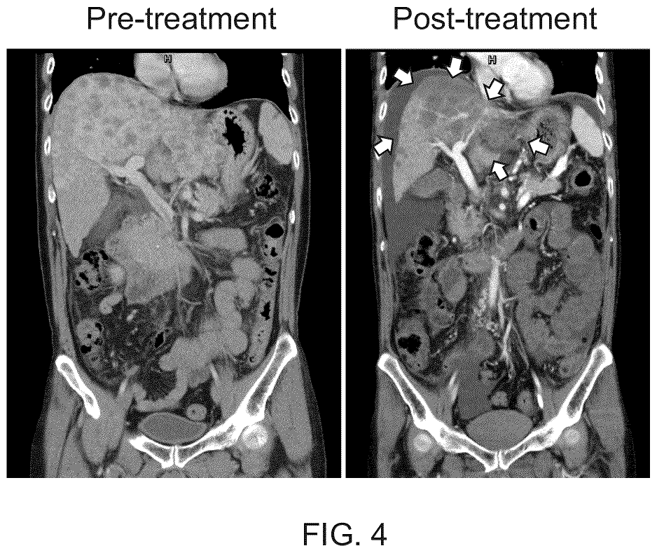

As shown in FIG. 3 and FIG. 4, coronal CT images reveal that, compared to the original infiltrative tumor region (pre-treatment, left panels), the tumor parenchyma of Patient 2 exhibited distinct mottled gray-black areas after four treatment sessions (arrows, right panels), indicating extensive tumor necrosis and a marked reduction in tumor size. These results demonstrate that the method described in the present invention effectively induces tumor destruction and shrinkage in hepatocellular carcinoma, thereby showing therapeutic efficacy against liver cancer.

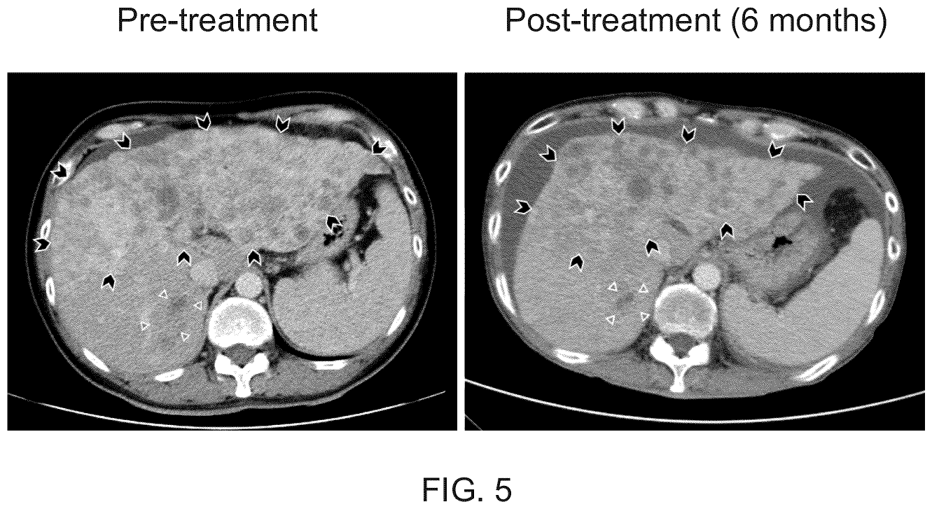

As shown in FIG. 5, cross-sectional CT images demonstrate that, compared to the original infiltrative tumor areas (pre-treatment, left panel), the upper tumor region in Patient 2 exhibited substantial reduction following five treatment sessions (indicated by black arrows with white borders, right panel). The lower tumor region (indicated by gray arrows with white borders) also showed notable regression. These observations support the therapeutic efficacy of the method described in the present invention for the treatment of liver cancer.

### 2.2 PIVKA-II Measurement

PIVKA-II (Protein Induced by Vitamin K Absence or Antagonist-II) is an abnormal prothrombin produced by malignant hepatocytes due to impaired carboxylation. Clinically, it serves as a tumor marker for liver cancer, assisting in diagnosis, monitoring treatment response, and evaluating disease progression or recurrence.

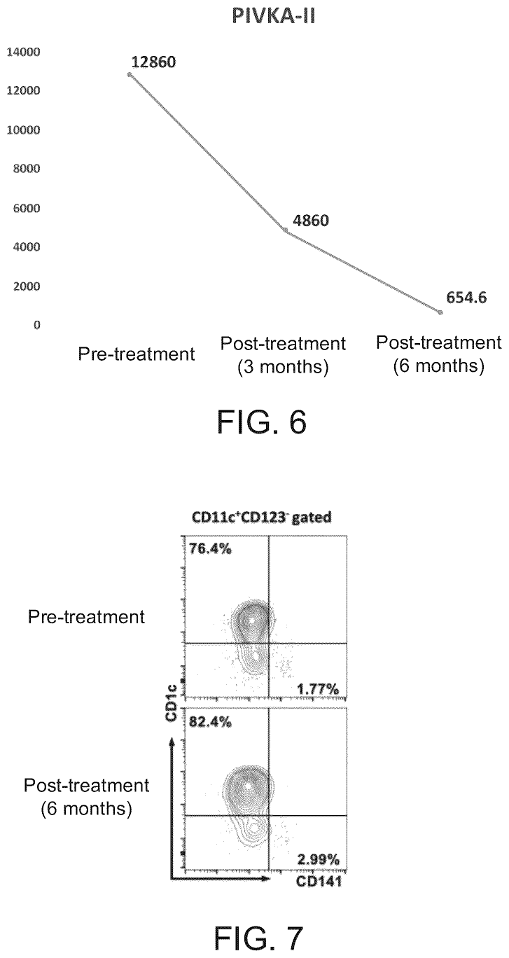

As shown in FIG. 6, Patient 2 presented a baseline PIVKA-II level of 12,860 mAU/mL prior to treatment. After three months of therapy, the level decreased to 4,860 mAU/mL and further declined to 654.6 mAU/mL after six months of treatment. This marked reduction in PIVKA-II levels demonstrates the therapeutic efficacy of the method described in the present invention for treating liver cancer.

### 2.3 Immune Cell Analysis

In this Example, dendritic cells, CD4⁺ T cells, and CD8⁺ T cells from Patient 2 were further analyzed by flow cytometry to evaluate the effect of the method described in the present invention on the patient's immune cells. Following pre-gating on CD11c⁺CD123⁻ cells, activated dendritic cells were identified by CD1c⁺CD141⁻ markers. Functional activated T cells were characterized using activation markers CD38 and HLA-DR in patients at baseline and after six months of treatment.

As shown in FIG. 7, prior to the treatment, activated dendritic cells in Patient 2 accounted for approximately 76.4% of the total dendritic cell population (upper panel). In contrast, after six (6) months of treatment, the activated dendritic cells increased to approximately 82.4% (lower panel). These results indicate that the method described in the present invention can effectively increase the proportion of activated dendritic cells in the patient.

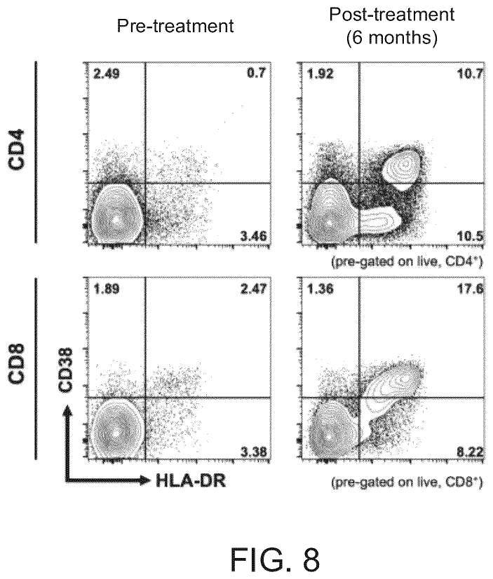

As shown in FIG. 8, prior to the treatment, activated CD4⁺ T cells in Patient 2 accounted for approximately 0.7% of the total CD4⁺ T cell population, and activated CD8⁺ T cells in Patient 2 accounted for approximately 2.47% of the total CD8⁺ T cell population, indicating that the proportion of activated T cells before treatment was quite low. In contrast, after six (6) months of treatment, the activated CD4⁺ T cells and the activated CD8⁺ T cells increased to 10.7% and 17.6%, respectively. These results indicate that the method described in the present invention can effectively increase the proportions of activated CD4⁺ T cells and activated CD8⁺ T cells in the patient.

### 2.4 Detection of Circulating Tumor Cells (CTCs)

In this Example, changes in circulating tumor cells were further analyzed to evaluate the therapeutic efficacy of the method described in the present invention. CTCs are tumor cells that have detached from the primary tumor and entered the bloodstream. Quantification of CTCs can be used for early cancer diagnosis as well as for monitoring the effectiveness of subsequent treatments.

Since the density of CTCs is similar to that of mononuclear cells (approximately 1.055 to 1.08 g/mL), the leukapheresis method used in the present invention not only separates mononuclear cells but also collects CTCs. Therefore, compared to peripheral blood sampling, leukapheresis can more effectively concentrate CTCs, enabling more accurate monitoring of cancer treatment efficacy.

Samples obtained by leukapheresis from Patient 2 were collected in blood collection tubes containing the anticoagulant K₂EDTA for quantification of CTCs using an automated microfluidic rare cell detection system (MiSelect R II System, MiCareo Taiwan Co., Ltd., Taipei, Taiwan). This system detects cells labeled with fluorescent markers via a microfluidic chip and identifies them using a fluorescent microscope. The quantitative results are presented in Table 2 below.

TABLE 2
Quantitative results of circulating tumor cells

| | Sample | | Number of |
|---|---|---|---|
| | volume | Number | PD-L1(+) |
| Detection time point | (mL) | of CTCs | CTCs |
| On the day of first treatment | 8 | 11 | 8 |
| On the day of second treatment/ | 8 | 8 | 6 |
| The condition after the first treatment | | | |
| On the day of third treatment/ | 8 | 3 | 2 |
| The condition after the second | | | |
| treatment | | | |
| On the day of fourth treatment/ | 8 | 2 | 2 |
| The condition after the third treatment | | | |
| On the day of fifth treatment/ | 8 | 1 | 1 |
| The condition after the fourth treatment | | | |

The results show that following the treatments described in the present invention, Patient 2 exhibited a progressive decrease in CTCs. Similarly, the number of PD-L1 (+) CTCs also declined over time, indicating an improvement in the patient's liver cancer condition. These findings demonstrate that the method described in the present invention has therapeutic efficacy against liver cancer.

The method of the present invention has the following advantages.

First, through the use of leukapheresis, autologous immune cells, specifically dendritic cells (DCs) and T cells, are rapidly isolated and concentrated, then reinfused directly into the target tumor site within a short period (within 2~12 hours). Conventional DC-based immunotherapies often face limitations due to difficulties in tumor antigen acquisition and challenges related to tumor heterogeneity and mutations. In contrast, our approach offers superior therapeutic immediacy and enhances the fidelity of antigen presentation at the tumor site.

Given that patients with advanced HCC often present with compromised physical status, this approach circumvents the need for prolonged in vitro cytokine-based stimulation and expansion required by other cell-based therapies. As a result, through this intervention method, it not only preserves the functional viability of the reinfused immune cells but also minimizes the risk of non-specific immune-mediated damage and reduces potential adverse effects, such as reactions to exogenous cytokines or allogeneic immune rejection.

In addition, as hepatic tumors primarily receive blood supply via the hepatic artery, our novel approach mimics hepatic artery infusion used in transarterial chemoembolization (TACE). We alternate the infusion of freshly isolated, concentrated autologous immune cells with immune checkpoint inhibitors (IO) directly into the hepatic artery at the tumor site. This method offers two major advantages:

1. It prevents therapeutic immune cells from being diluted by systemic circulation, enhancing the local concentration and efficacy at the tumor site.

2. Intra-arterial infusion of immune checkpoint inhibitors within the tumor microenvironment enhances the anti-tumor activity of CD8+ T cells. Therefore, this dual hepatic artery delivery of immune cells and IO therapy simultaneously augments both in situ DC vaccination effect and in situ anti-tumor T-cell responses.

To date, no existing therapy has integrated such a multifaceted and synergistic strategy to treat advanced liver cancer.

Finally, the liver is the largest solid organ in the human body, with a unique microcirculatory structure where immune cells circulate and infiltrate slowly through sinusoidal spaces, contributing to immune surveillance and regulation. To leverage these physiological features, our therapy employs hepatic artery infusion of an ICI and autologous immune cells, differing from conventional peripheral intravenous administration. This novel approach employs a prolonged infusion period (>4 hours), calibrated to match hepatic physiological dilution rates, thereby maintaining a consistent therapeutic dose over time. By doing so, it optimizes the intratumoral retention and functional activity of both immune checkpoint inhibitors and adoptively transferred immune cells, ultimately enhancing their anti-tumor efficacy within the liver tumor microenvironment.

Given all the apparent advantages, we believe the synergic treatment effects of immune-oncology therapy and cellular therapy in our approach will illuminate a pathway of hope for liver cancer patients.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A method for treating liver cancer in a subject in need thereof, comprising:
administering a therapeutically effective amount of autologous immune cells to the subject; and
administering a therapeutically effective amount of an ICI to the subject;
wherein the therapeutically effective amount of the autologous immune cells and the therapeutically effective amount of the immune checkpoint inhibitor are administered to the subject via hepatic artery infusion.

2. The method according to claim 1, wherein the therapeutically effective amount of the autologous immune cells and the therapeutically effective amount of the immune checkpoint inhibitor are administered to the subject simultaneously.

3. The method according to claim 1, wherein a portion of the therapeutically effective amount of the autologous immune cells is administered to the subject first, the therapeutically effective amount of the immune checkpoint inhibitor is administered to the subject secondly, and then a remaining portion of the therapeutically effective amount of the autologous immune cells is administered to the subject.

4. The method according to claim 3, wherein the portion of the therapeutically effective amount of the autologous immune cells is one quarter to three quarters of the therapeutically effective amount.

5. The method according to claim 3, wherein the remaining portion of the therapeutically effective amount of the autologous immune cells is three quarters to one quarter of the therapeutically effective amount.

6. The method according to claim 1, wherein the therapeutically effective amount of the autologous immune cells is isolated through leukapheresis.

7. The method according to claim 6, wherein the leukapheresis is configured to isolate mononuclear cells having a density between 1.055 to 1.08 g/mL.

8. The method according to claim 6, wherein the therapeutically effective amount of the autologous immune cells is administered within 12 hours after the autologous immune cells are isolated.

9. The method according to claim 1, wherein the therapeutically effective amount of the autologous immune cells comprises dendritic cells and lymphocytes.

10. The method according to claim 1, wherein the therapeutically effective amount of the autologous immune cells comprises at least 1×10⁶ dendritic cells.

11. The method according to claim 1, wherein the therapeutically effective amount of the autologous immune cells comprises at least 1×10⁸ lymphocytes.

12. The method according to claim 1, wherein the therapeutically effective amount of the autologous immune cells comprises at least 1×10⁶ dendritic cells and at least 1×10⁸ lymphocytes.

13. The method according to claim 1, wherein the immune checkpoint inhibitor is a PD-L1 inhibitor.

14. The method according to claim 1, wherein the therapeutically effective amount of the immune checkpoint inhibitor comprises 1~50 mg/mL of PD-L1 inhibitor.

15. The method according to claim 1, wherein the therapeutically effective amount of the immune checkpoint inhibitor comprises 1~5,000 mg of PD-L1 inhibitor.

16. The method according to claim 1, wherein the subject has liver cancer.

17. The method according to claim 16, wherein the subject received radiotherapy prior to the administration of autologous immune cells and the immune checkpoint inhibitor.
